(19) United States
(12) Patent Application Publication
(10) Pub. No.: US 2023/0190518 A1
(43) Pub. Date: Jun. 22, 2023

(54) DEVICE AND METHOD FOR WEARABLE HEATING PACK

(71) Applicants: Charles Katrycz, Toronto (CA); Katherine Porter, Oakville (CA)

(72) Inventors: Charles Katrycz, Toronto (CA); Katherine Porter, Oakville (CA)

(21) Appl. No.: 18/069,852

(22) Filed: Dec. 21, 2022

(30) Foreign Application Priority Data
Dec. 21, 2021 (CA) 3143448

(51) Int. Cl. A61F 7/02

(52) U.S. Cl. CPC A61F 7/02; A61F 2007/0022

(57) ABSTRACT

A device and method for a wearable heat pack is presented. The heat pack provides a therapeutic application of heat or cold to a user using phase-change material to provide a desired temperature and is made of a soft and flexible material that is comfortable and pleasant to use, but is strong enough to be reused multiple times. The heat pack device includes a sealed silicone envelope. An infill is contained in the envelope and includes a phase-change material for providing a temperature range over a period of time. The method includes heating the heat pack device, placing the heat pack device in a pocket of a garment, and placing the garment on a user to position the heat pack device in contact with the user, wherein the heat pack device includes a phase change material and further wherein the heating creates a phase-change of the phase-change material.

The present application claims priority to Canadian Patent Application 3,143,448 filed on Dec. 21, 2021, presently pending, the contents of which are incorporated by reference.

### FIELD

The present disclosure relates generally to a heating pack, and more specifically, to a device and method for a wearable heating pack.

### BACKGROUND

Personal heating and cooling devices are useful for pain relief, injury treatment, soothing sore muscles, and other therapeutic applications. Traditional examples of such devices are electric heating pads that can be applied to a skin surface of a user. Heating/cooling packs may utilize gels or dry ingredients such as rice, flaxseed, or oatmeal, which are stored in a freezer or microwaved to provide a cold or hot pack, respectively. Instant hot and cold packs use a thermochemical reaction to release heat in an exothermic reaction or absorb heat in an endothermic reaction. Phase change materials have also been used for heating or cooling, depending on whether the phase change is exothermic or endothermic. What is lacking in the field is a personal heating/cooling device that provides a safe and comfortable temperature range for application to a user’s skin, and that is contained in a soft, flexible, durable, and non-toxic material.

### SUMMARY

In the present disclosure, a device and method for a wearable heat pack is provided. The heat pack provides a therapeutic application of heat or cold to a user. The heat pack uses phase-change material to provide a desired temperature and is made of a soft and flexible material that is comfortable and pleasant to use but is strong enough to be reused multiple times.

Thus by one broad aspect of the present invention, a heat pack device is provided that includes a sealed silicone envelope having an outside perimeter and an inside perimeter. An infill is contained in the envelope and includes a phase-change material for providing a temperature range over a period of time.

By a further aspect of the present invention, a method is provided for using a heat pack device. The method includes heating the heat pack device, placing the heat pack device in a pocket of a garment, and placing the garment on a user to position the heat pack device in contact with the user, wherein the heat pack device includes a phase change material and further wherein the heating creates a phase-change of the phase-change material.

A further understanding of the functional and advantageous aspects of the invention can be realized by reference to the following detailed description and drawings.

### BRIEF DESCRIPTION OF THE DRAWINGS

Embodiments disclosed herein will be more fully understood from the following detailed description taken in connection with the accompanying drawings, which form a part of this application, and in which:

FIG. 1 is a front planar view of a heat pack according to an embodiment of the present disclosure.

FIG. 2 is a back planar view of the embodiment of the heat pack illustrated in FIG. 1.

FIG. 3 is an exploded view of the embodiment of the heat pack illustrated in FIG. 1.

FIG. 4 is a cross-sectional view of the embodiment of the heat pack illustrated in FIG. 1.

FIG. 5 is a planar view of the embodiment of the heat pack illustrated in FIG. 1 and an exterior front view of a garment according to an embodiment of the present disclosure.

FIG. 6 is a planar view of the heat pack and interior front view of the garment of the embodiment illustrated in FIG. 5.

FIG. 7 is a planar view of the heat pack and exterior back view of the garment of the embodiment illustrated in FIG. 5.

FIG. 8 is a planar view of the heat pack and interior back view of the garment of the embodiment illustrated in FIG. 5.

### DETAILED DESCRIPTION

The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

The present invention has been shown and described in a preferred embodiment. It is recognized, however, that departures may be made within the scope of the invention and that obvious modifications will occur to a person skilled in the art. With respect to the above description, it is to be realized that the optimum dimensional relationships for the parts of the presented invention, including variations in size, materials, shape, form, function, and manner of operation, assembly, and use, are deemed readily apparent and obvious to one skilled in the art, and all equivalent relationships to those illustrated in the drawings and described in the specifications are intended to be encompassed by the present invention.

Therefore, the foregoing is considered as illustrative only of the principles of the present invention. Further, since numerous modifications and changes will readily occur to those skilled in the art, it is not desired to limit the present invention to the exact construction and operation shown and described, and accordingly, all suitable modifications and equivalents result in falling within the scope of the invention.

A heat pack is disclosed that provides a therapeutic application of heat or cold to a user. The heat pack uses phase-change material to provide a desired temperature and is made of a soft and flexible material that is comfortable and pleasant to use, but is strong enough to be reused multiple times.

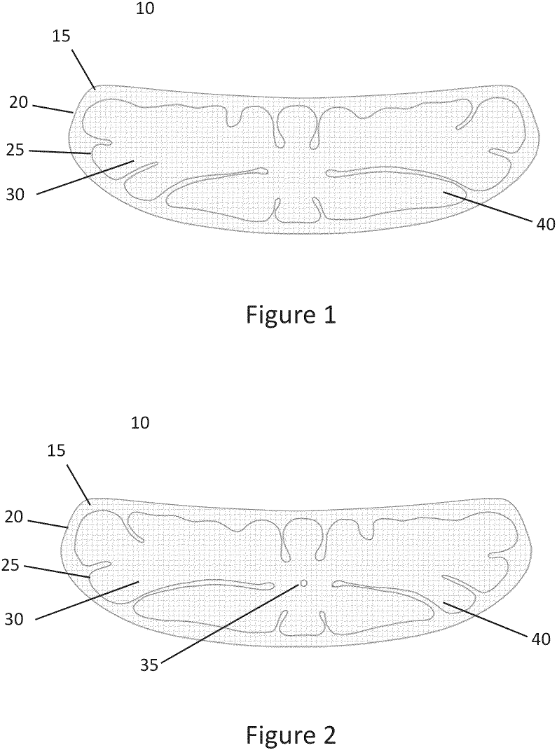

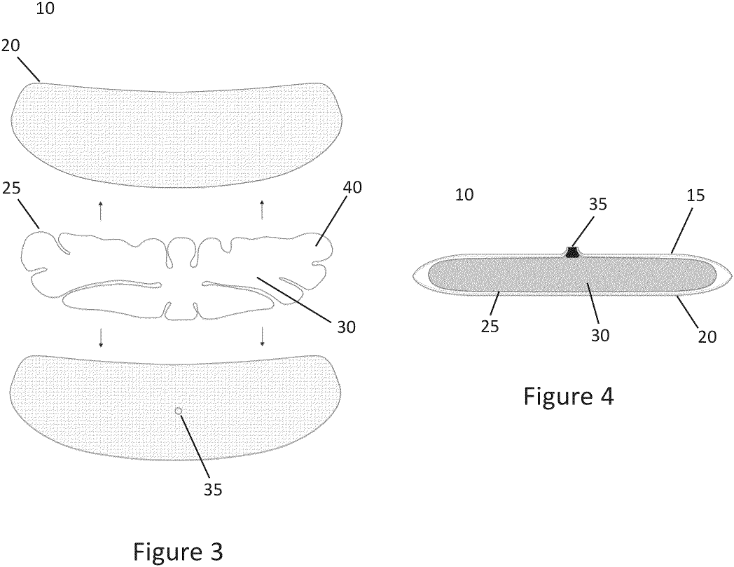

Referring to FIGS. 1 to 4, the heat pack 10 includes a sealed silicone envelope 15, which has an outside perimeter 20 and an inside perimeter 25. The silicone envelope 15 is seamless, which also contributes to the comfort and safety of using the heat pack 10. The silicone envelope 15 is filled with an infill 30. The infill 30 is provided into the silicone envelope 15 through an orifice 35. The infill 30 is made of a phase-change material that allows the heat pack 10 to provide a desirable temperature range.

In the application of heat to a user’s body, the material makeup of the envelope 15 is important because it comes in close contact with the user’s skin. The silicone envelope 15 is skin-safe and does not absorb or leach any harmful chemicals, whereas many petroleum-based plastics contain toxic chemicals (e.g., bisphenols) and so are not appropriate for use against human skin. The silicone envelope 15 is soft, with no sharp edges or corners, and conforms to the skin surface. Coarse or hard materials should not be used to avoid discomfort or irritation to the skin. The silicone envelope 15 is tough and durable to avoid puncture or tearing, which could cause rupture of the inner material onto the skin, and potentially cause burns. Welded seams are avoided to prevent the inadvertent inclusion of weak points in the envelope 15 that might tear. The silicone envelope 15 performs the above-mentioned criteria (skin-safe, soft and flexible, tough and durable) under a suitable temperature range. While the heat pack device 10 is not intended to be applied to the skin while heated above 100 C, the method of heating may cause the silicon envelope 15 to reach such a temperature prior to application to the skin, and the durability of the envelope is not lessened, even if the envelope temperature is increased to between 150 C-200C.

While many materials can meet any one of these requirements, few materials meet them all. There are virtually no plastics that are skin safe, soft, and capable of withstanding high temperatures (>200 C). Moreover, few techniques exist to create seamless inner cavities within the envelope 15, and so any lamination techniques that would normally be used in creating such an envelope using plastics will necessarily introduce seams with sharp edges and vulnerable points of weakness.

Silicone rubber is uniquely well-suited for such on-the-body applications. Silicone comes in skin-safe, food-grade, and medical-grade specifications and is often used in medical applications, having an inert surface chemistry that allows it to be used on the skin’s exterior surface and even surgically inserted within the body. Silicone comes in a wide range of hardnesses, including Shore-A 12-50, which includes ideal hardnesses for on-the-body applications. It is soft to the touch and easily conforms to the contours of the body. Silicone is tough and durable and can elongate by 400-600% before breaking. It can be processed in such a way as to include no internal seams (Katrycz U.S. Pat. No. 10245762, CA patent no. 2,921,441). Silicone has an operating temperature range of -60 C to 230C, making it resilient to overheating, for example, in a microwave oven.

The silicone envelope 15 with an outer perimeter 20 and an inner perimeter 25 may be made by blowing air into a liquid silicone volume to envelope a large air cavity in silicone. Silicone is a thermoset elastomer, and so after mixing the two-part epoxy resin together, it will cure into solid rubber, provided that the ambient temperature is not inhibiting, and that there is no cure-inhibiting chemistry present. The solidification process can proceed without ventilation, differing from other resins, which may need to evaporate a solvent in order to cure. This allows the silicone to be injection molded in unique ways, including the air-assisted method of injection molding that produces a branching pattern along the inner perimeter 25, forming branched channels 40, as described in Patent 10245762. In another embodiment, traditional silicone processing can be used. This embodiment uses two-part casting and laminating to create a sealed enclosure. This process requires separate molding of two sides of the silicone envelope 15. The sides are molded in silicone, and then the two sides are brought into contact for bonding.

In order to provide the silicone envelope 15 with heat-carrying capacity, the silicone envelope is filled with a phase change material (PCM) infill 30. PCMs are well-studied for their ability to absorb and emit heat while maintaining a constant temperature. The heat of fusion, for example, is the latent heat absorbed/emitted by a mass of PCM to change its phase to/from liquid from/to solid. This heat is extremely useful in a wearable application, as a PCM within the silicone envelope 15 can be applied to the body to deliver heat. To do so, the sealed silicone envelope 15 containing a PCM infill 30 (heat pack 10) is heated. Heat can be applied to a heat pack 10 by way of immersion in hot water, baking in an oven, or heating in a microwave oven. Once the PCM infill 30 has melted to a liquid form, the heat pack 10 can be removed from the heat source. Once the heat pack 10 is at a suitable temperature of between 65C-90C, it can be applied to the body to deliver heat. Because the PCM solidifies at the solidification temperature (liquid-solid transition temperature), the heat of fusion will be released into the body from the PCM infill 30 through the silicone envelope 15 and through the skin. This heat can then contribute to pain relief by increasing blood flow to the affected area.

Silicone is naturally a heat-insulating material, having a low heat conductivity coefficient. However, if the silicone layer of the silicone envelope 15 is thin enough, it can easily conduct heat from the PCM infill 30 to the skin. To make silicone thin, care has to be taken to ensure that the silicone membrane is still sufficiently durable as to prevent leaks. To accomplish this, a fabric or fiber network can be introduced into the silicone membrane to reinforce the membrane of the silicone envelope 15. Through co-molded injection molding processes, one can incorporate stretchy nylon meshes into the silicone. The incorporation of nylon mesh into the silicone membrane provides the additional strength of the nylon against tearing, allowing the silicone membrane to be made thinner. Such a combination can be used to produce durable layers of 0.5 mm thickness.

The air-assisted method of injection molding described in Patent 10245762 allows for the production of branched channels 40 within the silicone envelope 15. Such branched channels 40 allow for the heat pack 10 to maintain a slim profile, reducing bulging by connecting the anterior and posterior faces of the envelope 15 with interconnecting webs of silicone, while also allowing for a single source of injection for filling the envelope with PCM. Such a branched structure is reminiscent of many organs, including the lungs, kidneys, and liver. This type of architecture is beneficial to the structure of the heat pack 10 and the conduction of heat from the pack. The structure of the branched channels 40 allows for large surface areas of contact between the heat pack 10 and the skin, enabling heat transfer.

PCMs can take advantage of a variety of phase transformations. The most relevant to the present application is the liquid-solid phase transformation. It is generally the case that liquid-to-solid transitions liberate the most enthalpy of fusion, as compared to solid-solid transitions, because of the great reduction of entropy within the volume as the solid phase crystallizes out of a liquid melt. This heat is of great benefit in the present application, as it is the primary storage vehicle for the heat that is applied to the heat pack 10, and that is released from the heat pack. Moreover, as the initial state of the PCM on application to the body is the liquid phase, the silicone envelope 15 is most highly conforming to the body in this state. Thus the heat pack 10 with liquified PCM infill 30 can be closely applied to the body with great comfort. As the PCM crystallizes and solidifies, the heat pack 10 then molds to the shape of the body. Thus, even though the transition leaves the heat pack 10 in an end state that has a degree of stiffness to it, the heat pack has taken on the form of the body where it was applied and thus is customized to fit on the body.

The temperature at which point the PCM releases most of its heat, and transitions from liquid to solid, can be tailored to application by choosing the PCM carefully. An ideal temperature for heat delivery to the skin is between 40C-100 C, depending on the conductivity of the interface between the PCM and the human skin. An ideal enthalpy of fusion is greater than 200 kJ/kg.

In an embodiment, organic waxes, such as paraffins and beeswax, are used as the PCT infill 30, having an ideal melting point between 46-68 C, and 62-65 C, respectively. The large heat of fusion of 242 kJ/kg for beeswax is superior to that of paraffin at 220 kJ/kg. Beeswax comes from a natural and renewable source, is skin-safe, and comes in food-grade designation, making it an ideal candidate for on-the-body applications.

Other PCMs that make good on-the-body heating candidates include fatty acids, sugar alcohols, and natural resins.

Another advantage of the silicone envelope 15 is that it is semi-permeable to organic oils. This allows for the inclusion and mixture of the inner PCM infill 30 with essential oils and topical oil treatments, which over time, are slowly released through the silicone envelope. This leaching effect has been an unwanted effect in other PCM applications and has often limited the selection of envelope materials. PCMs can be mixed with castor oil, mint oil, lavender, and vegetable oils. By mixing beeswax with an unsaturated fat that is liquid at room temperature, for example, olive or vegetable oil, the beeswax becomes softer at room temperature. This is useful in producing a softer end-state of the heat pack 10 than would be possible with pure beeswax. Mix ratios can range from 10-90% vegetable oil by weight in such mixtures. Mixing unsaturated fats like vegetable oil with beeswax will also lower the melting point of the mixture and reduce the enthalpy of fusion.

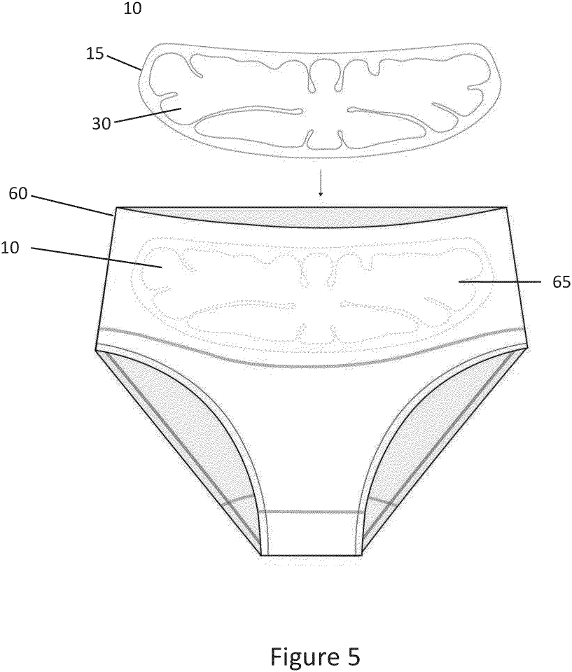

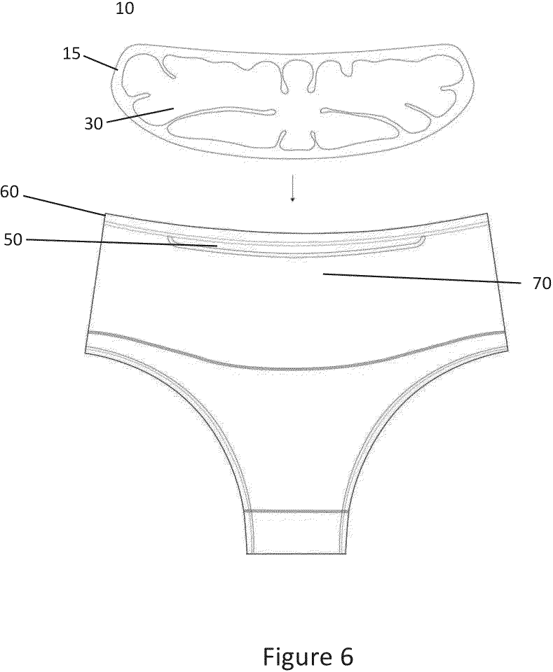

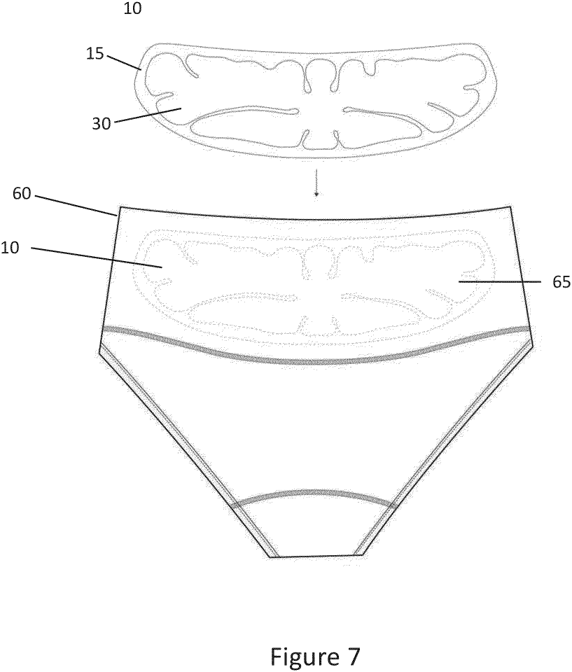

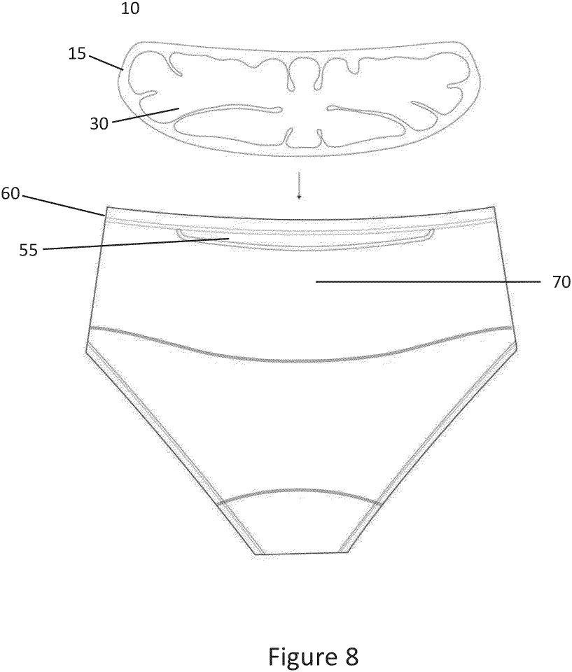

The heat pack 10 can be used on its own with the silicone envelope 15 applied directly against the skin, or it can be inserted into a fabric pocket 50, 55 incorporated within a garment 60, as shown in example embodiments of FIGS. 5-8. The garment 60 provides support for the heat pack 10 and ensures the heat pack is positioned where it is needed most. In one embodiment of the invention, the garment 60 is a pair of underwear with a front pocket 50 and a back pocket 55. Said garment 60 allows the heat pack 10 to be applied directly to the lower abdomen and lower back, to alleviate pelvic pain. In another embodiment, the pockets 50, 55 can be incorporated into a pair of leggings to apply the heat pack to the thighs to alleviate painful cramps brought on by menstruation.

The pockets 50, 55 house the heat pack 10 against point-specific locations of the body that are proven to be susceptible to the experience of menstrual pain. In one embodiment, this may include the lower abdomen, the posterior lower back, and lateral lower hip. Other embodiments may include other locations. The pockets 50, 55 have a different technical composition than the garment 60, and use layered/laminated technical textiles to insulate, direct and dissipate heat from the heat-producing heat pack 10, towards the body. In other embodiments, the technical pockets 50, 55 may be used to insulate, direct and dissipate cold to the body.

The pockets 50, 55 each have an exterior layer 65 and an interior layer 70, relative to the user wearing the garment 60, to enhance the functionality of the heat pack 10. The exterior layer 65 provides thin, soft, conformable thermal insulation of the heat produced by the heat pack 10. This function ensures heat is not lost to the environment and helps maximize the longevity of the heat-producing heat pack 10 and efficiently directs the contained heat into the body. In one embodiment, the exterior layer 65 consists of an infrared reflective coating on the fabric, combined with a thin, insulative material to prevent heat loss due to radiation and conduction. In one embodiment, this exterior layer 65 is structured similarly to performance thermal garments used for winter activity which incorporate a reflective coating on a stretch cotton or wool.

The interior layer 70 of the pocket 50, 55 is capable of distributing heat from the heat pack to the body via conduction. In an embodiment, the interior layer 70 is made of a thin, soft, flexible textile material. The material can partially insulate the heat to provide a buffer that evenly distributes the heat to the desired region and mitigates discomfort caused by overheating. In another embodiment, the interior layer 70 is made of conductive materials, such as copper fibers or graphene. Such layers will increase the conductivity between the heat pack 10 and the skin.

In one embodiment, the interior layer 70 in contact with the skin is capable of absorbing and wicking away water and moisture caused by sweat or condensation. This material is soft and comfortable on the body, and integrates into the garment to provide a technical region, or thermal window, with thermal and moisture properties that are preferred.

To use the heat pack 10 it must first be heated. The process of heating can use dry forms of heat such as a microwave oven or baking in a conventional or solar oven, or the heat pack 10 can be boiled or immersed in hot water. In one embodiment, the heat pack 10 is rolled up so that it fits into a mug or thermos and then immersed in boiling hot water by pouring hot water into the vessel, as one would do to steep a tea bag. The heat from the water is transferred into the heat pack 10, melting the PCM infill 30 and causing the heat pack 10 to charge up with heat. The heat pack 10 can then be removed from the mug or water-containing vessel and applied to the body either directly or inserted into the pocket 50, 55. In another embodiment, the heat pack 10 is placed in the microwave on a dish. The microwave is set on high for 1-3 minutes to heat the heat pack 10. This microwave heating will melt the PCM infill 30, thus charging the heat pack 10 with heat. Upon completion of the microwave step, the heat pack 10 can be removed from the microwave and applied directly to the skin or inserted into pockets 50, 55. In another embodiment, the heat pack 10 can be heated using a resistive electrical heating element. The heating element is brought into thermal contact with the heat pack 10 and charges the PCM infill 30 through electrical resistive heating.

What is claimed is:

1. A heat pack device comprising:
a sealed silicone envelope having an outside perimeter and an inside perimeter; and
an infill contained in the envelope, the infill comprising a phase-change material for providing a temperature range over a period of time.

2. The heat pack device of claim 1, wherein the silicone envelope comprises at least one of a reinforcing fabric or a reinforcing fiber.

3. The heat pack device of claim 1, wherein the silicone envelope comprises a seamless structure, for protecting against ruptures.

4. The heat pack device of claim 1, wherein the silicone envelope inside perimeter comprises a branching pattern.

5. The heat pack device of claim 1, wherein the phase-change material comprises a liquid to a solid phase change.

6. The heat pack device of claim 1, wherein the phase-change material temperature range provides heat.

7. The heat pack device of claim 1, wherein the phase-change material comprises an organic wax.

8. The heat pack device of claim 7, wherein the organic wax comprises at least one of: beeswax; soy wax; carnauba wax; and pine resin.

9. The heat pack device of claim 7, wherein the infill further comprises an essential oil.

10. The heat pack device of claim 9, wherein the essential oil comprises at least one of: castor oil; mint oil; and vegetable oil.

11. The heat pack device of claim 1, further wherein the heat pack is housed in a pocket of a garment.

12. The heat pack device of claim 11, wherein the pocket comprises a layered insulating texture.

13. The heat pack device of claim 11, wherein the garment has a plurality of pockets, each pocket housing a heat pack.

14. The heat pack device of claim 13, wherein the garment comprises an undergarment for applying the heat pack to a user abdomen and lower back.

15. A method for using a heat pack device comprising:
heating the heat pack device;
placing the heat pack device in a pocket of a garment; and
placing the garment on a user to position the heat pack device in contact with the user;
wherein the heat pack device comprises a phase change material, and
further wherein the heating creates a phase-change of the
phase-change material.

16. The method of claim 15, wherein heating the heat pack device comprises any one of:
immersing the device in boiling water;
microwaving the device;
placing the device in an oven; and
heating the device with an electrical element.

17. The method of claim 16, wherein immersing the device in boiling water comprises:
rolling the device into a compact size;
placing the device into a vessel; and
adding boiling water to the vessel.
